(19) United States
(12) Patent Application Publication
(10) Pub. No.: US 2026/0232331 A1
(43) Pub. Date: Aug. 13, 2026

(54) PROPHYLACTIC METHOD OF REDUCING ACROMIAL STRESS FRACTURE INCIDENCE AND KIT FOR PERFORMING THE SAME

(71) Applicant: Arthrex, Inc., Naples, FL (US)

(72) Inventors: Kevin Gallen, Naples, FL (US); Alyssa Morgan, Naples, FL (US); Steven Schewe, Naples, FL (US); Patrick Denard, Medford, OR (US); Brian C. Werner, Charlottesville, VA (US)

(73) Assignee: Arthrex, Inc., Naples, FL (US)

(21) Appl. No.: 19/533,630

(22) Filed: Feb. 9, 2026

Related U.S. Application Data
(60) Provisional application No. 63/756,985, filed on Feb. 11, 2025.

(51) Int. Cl. A61B 17/17; A61B 17/56; A61B 34/10; A61F 2/46

(52) U.S. Cl. CPC A61B 17/1778; A61B 17/1703; A61B 34/10; A61F 2/4612; A61B 2017/564; A61B 2034/107

(57) ABSTRACT

A method of prophylactically reducing acromial fracture incidence may include locating a lateral border of an acromion of an intact scapula. A posterior entry location may be located on the acromion at a first predefined distance from a posterolateral corner of the acromion. A first incision may be made at the posterior entry location. A first pin may be inserted into the acromion at the posterior entry location and guided toward a scapular spine of the intact scapula. An anterior entry location may be located on the acromion, which is a second predefined distance from the posterior entry location. A second incision may be made at the anterior entry location. A second pin may be inserted into the acromion at the anterior entry location and guided toward the scapular spine of the intact scapula. The first and second pins are trimmed at the edge of the acromion.

### CROSS-REFERENCE TO RELATED APPLICATION

This application claims priority under 35 U.S.C. § 119 (e) and the benefit of U.S. Provisional Application No. 63/756,985 entitled PROPHYLACTIC METHOD OF REDUCING ACROMIAL STRESS FRACTURE INCIDENCE, filed on Feb. 11, 2025, the entire disclosure of which is incorporated herein by reference.

### BACKGROUND

The present disclosure generally relates to a method for reducing or preventing acromial fractures, and more particularly to reducing or preventing acromial fractures after a reverse shoulder arthroplasty.

Stress may be applied to an acromion of the scapula in response to surgical procedures, such as replacements, arthroplasty procedures, and reverse shoulder arthroplasty procedures, or in response to certain injuries. The bone portions on opposing side of the fractures are often fixed together using bulky plates implanted into the patient. In various implementations, the disclosure provides for devices and methods that may assist in reducing or preventing the occurrence or likelihood of acromial stress fractures to improve patient outcomes.

### SUMMARY

According to at least one aspect of the present disclosure, pins may be used for prophylactically treating acromial stress fractures and/or for reducing the likelihood of acromial fracture development. The pins may be inserted into intact bone to reinforce the bone and reduce the likelihood of an acromial stress fracture developing. In certain aspects, a method of prophylactically reducing acromial fracture incidence in response to a reverse shoulder arthroplasty may include locating a lateral border of an acromion of an intact scapula and locating a posterior entry location on the acromion at a first distance from a posterolateral corner of the acromion. A first incision may be made at the posterior entry location. A first pin may be inserted into the acromion at the posterior entry location and guided toward a scapular spine of the intact scapula. An anterior entry location on the acromion may be located. The anterior entry location may be a second distance from the posterior entry location. A second incision may be made at the anterior entry location, and a second pin may be inserted into the acromion at the anterior entry location and guided toward the scapular spine of the intact scapula. A guide may be utilized to form pilot holes with a drill and/or guide the pins into the bone. The first and second pins may be trimmed at an edge of the acromion, remaining in the intact scapula.

A surgical kit can be utilized to perform the method of reinforcing the acromion or reducing acromial fracture likelihood. The surgical kit may include the first and second pin assemblies having the first and second pins configured to remain in the bone and shafts selectively coupled to the pins. The kit may also include a drill guide. The drill guide may be configured to hook onto the acromion, which can assist with positioning the drill guide. The drill guide may include a foot to engage the bottom of the acromion and may extend around an edge of the acromion and over the top of the acromion. The drill guide may include an aiming point that may be adjustable based on patient anatomy. The drill guide may be utilized for forming pilot holes and/or guiding the pin assemblies into the bone.

The kit may also include a plate, which may assist the pins in supporting the acromion. The plate may have tabs that extend around the lateral border of the acromion and include apertures for receiving the pins. The plate may be secured to the bone via locking fasteners and/or a suture cerclage. The plate may be biomechanically optimized and contoured to the acromion, while minimizing the profile of the implanted components. The method may be performed with the pins alone or in combination with the plate.

These and other features, objects, and advantages of the present disclosure will become apparent upon reading the following description thereof together with reference to the accompanying drawings.

### BRIEF DESCRIPTION OF THE DRAWINGS

FIG. 1 is a schematic diagram of a kit for reducing acromial stress fracture development including pins, a drill guide, an aiming guide, a cutting device, a plate, and a driver, according to the present disclosure;

FIG. 2 is a side perspective view of a pin assembly with a trimmable pin with snap-off features, according to the present disclosure;

FIG. 3 is a schematic diagram of an intact scapula and natural glenohumeral joint, according to the present disclosure;

FIG. 4 is a schematic diagram of an intact scapula and artificial glenohumeral joint after a reverse shoulder arthroplasty, according to the present disclosure;

FIG. 5 is a schematic top perspective view of an intact scapula indicating fracture types according to the Levy classification illustrated, according to the present disclosure;

FIG. 6A is a schematic side perspective view of pins implanted in an intact acromion, according to the present disclosure;

FIG. 6B is a schematic top perspective view of an intact scapula with pin assemblies inserted into the intact scapula and shafts extending from an edge of an acromion, according to the present disclosure;

FIG. 6C is a schematic side elevational view of pins implanted in an intact acromion, according to the present disclosure;

FIG. 7 is a schematic diagram of a drill guide positioned on an acromion, according to the present disclosure;

FIG. 8 is a flow diagram of a method of prophylactically reducing a risk of acromial fracture incidence, according to the present disclosure;

FIG. 9 is a flow diagram for prophylactically pinning an intact scapula, according to the present disclosure;

FIG. 10 is a side perspective view of a plate that can be utilized with pins implanted in an intact acromion, according to the present disclosure;

FIG. 11A is a schematic top view of pins inserted into an intact acromion and engaging a plate, according to the present disclosure;

FIG. 11B is a schematic posterior view of pins inserted into an intact acromion and engaging a plate, according to the present disclosure;

FIG. 12 is a schematic diagram of a tab of a plate and a trimmable pin assembly configured to engage the tab, according to the present disclosure; and

FIG. 13 is a schematic view of a plate with a suture cerclage, according to the present disclosure.

### DETAILED DESCRIPTION

In the following description, reference is made to the accompanying drawings, which show specific implementations that may be practiced. Wherever possible, the same reference numbers will be used throughout the drawings to refer to the same or like parts. It is to be understood that other implementations may be utilized and structural and functional changes may be made without departing from the scope of this disclosure.

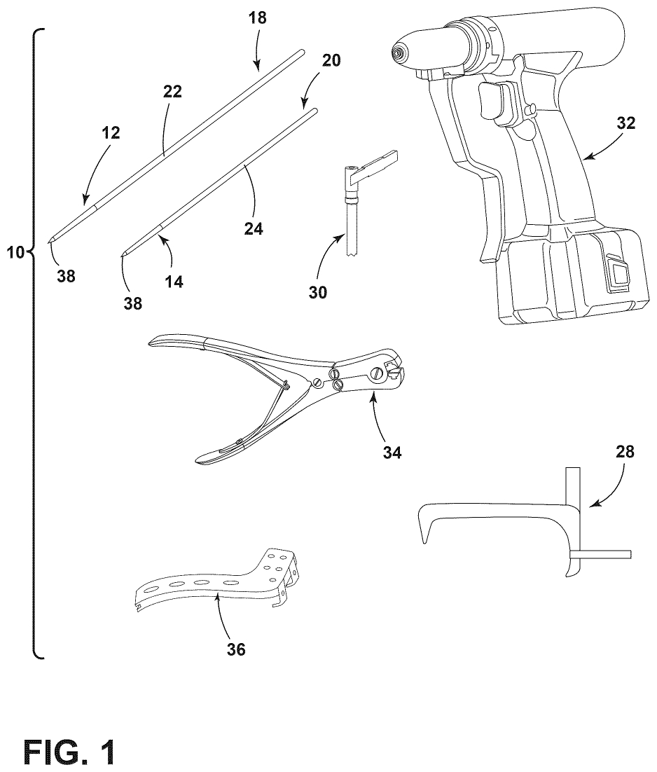

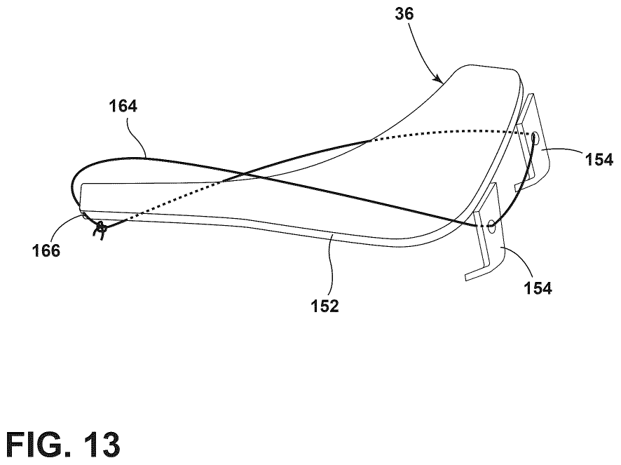

Referring to FIGS. 1-13, an assembly or kit 10 for reducing or preventing acromial stress fractures may include a first pin 12 and a second pin 14 for insertion into an acromion A of an intact scapula S. The first and second pins 12, 14 may be part of pin assemblies 18, 20 that are configured to be driven or inserted into the bone toward a scapular spine SS of the intact scapula S. The first and second pins 12, 14 may be inserted as a prophylactic measure for reducing or preventing acromial fractures that can result from certain procedures or injuries. The pins 12, 14 may be inserted into the acromion A and remain in the scapula S to provide additional support or reinforce the acromion A to reduce the likelihood of the acromial fracture developing.

Referring to FIG. 1, the kit 10 may include the first and second pins assemblies 18, 20 including the first and second pins 12, 14 with shafts 22, 24 coupled thereto, respectively, as well as instrumentation for inserting the pins 12, 14 in the bone. For example, the kit 10 may include a directional drill guide 28, which can assist in directing the pins 12, 14 along predefined paths into the intact bone. The directional drill guide 28 may be used for inserting the pins 12, 14 and may also be used with a drill for forming pilot or guide holes in the bone. The drill guide 28 may also be utilized for guiding insertion of the pins 12, 14. The kit 10 may include one or more aiming guides 30 for positioning the pins 12, 14 in the acromion A. The aiming guide 30 illustrated in FIG. 1 may be considered a soft tissue guide. The kit 10 may also include an insertion device or driver 32 for inserting the pins 12, 14 into the bone, a cutting or trimming device 34 for shortening the length of the pins 12, 14, and/or a device for repositioning the pins 12, 14. The kit 10 may include any instrumentation for determining pin 12, 14 positioning and assisting with inserting or adjusting the pins 12, 14. Further, in certain aspects, the kit 10 may include a plate 36 that can engage the acromion A and be secured by the pins 12, 14.

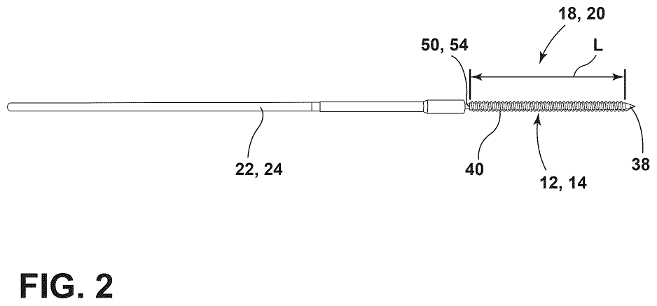

Referring still to FIG. 1, as well as FIG. 2, different types of pin assemblies 18, 20 may be utilized depending on the patient, the procedure, the area of the acromion at increased risk of fracture, the anatomy, etc. The pin assemblies 18, 20 utilized in a single procedure may be substantially identical, or different pin assemblies 18, 20 may be utilized in a single procedure. The pin assemblies 18, 20 may include the shafts 22, 24 coupled with the first and second pins 12, 14, respectively. The shafts 22, 24 may be selectively coupled to the pins 12, 14 to assist with inserting the pins 12, 14 into the acromion A and may be removable from the pins 12, 14 to leave the pins 12, 14 implanted into the bone. The shafts 22, 24 may be elongated structures that form extensions of the pins 12, 14 for inserting and guiding the pins 12, 14.

The pins 12, 14 may be constructed of rigid material, such as, for example, stainless steel or titanium, to provide increased strength or support, while not significantly bending or deforming during the insertion process. The pins 12, 14 may be retained in the bone to provide structural reinforcement, which reduces the likelihood of a fracture from the increased stress from a procedure. The stiffness of the pins 12, 14 may allow the pins 12, 14 to act as “reinforcement bars” in the bone against various stresses and forces applied to the bone.

The pins 12, 14 may be elongated and narrow implants. The pins 12, 14 may be configured to extend at least partially across the scapula S. The pins 12, 14 may taper/narrow at an insertion end 38 and maintain a consistent diameter/width for the remainder of the pins 12, 14. The tapered insertion end 38 may assist with driving the pins 12, 14 into the bone. Alternatively, the pins 12, 14 may have a consistent diameter/width along the length thereof, including the insertion end 38.

The size of the pins 12, 14 may depend on the patient. For example, a patient with larger anatomy may utilize pins 12, 14 with a larger diameter and/or a longer length to provide increased support. In comparison, a patient with a smaller anatomy may utilize pins 12, 14 with a smaller diameter and/or a shorter length. This may provide support to the bone without weakening the bone due to pins 12, 14 that are too large for the anatomy. The pins 12, 14 utilized in a single procedure for the patient may have substantially similar diameters and lengths. Alternatively, the first pin 12 may have a different diameter and/or length than the second pin 14 based on the patient anatomy and position of the pins 12, 14 in the anatomy. In various aspects, the diameter of the pins 12, 14 may be between about 1.5 mm and about 3 mm. In non-limiting examples, the pins 12, 14 may be 2.4 mm threaded titanium pins 12, 14. The size of the pins 12, 14 may balance reinforcing the bone with reducing the weakening of the bone by forming larger holes from pins 12, 14 that are too large for the anatomy.

The pins 12, 14 may include features for being retained in the bone and reducing movement once implanted. For example, the pins 12, 14 may include a protruding structure 40 to find purchase in the bone, which may extend along a length of the pins 12, 14. The length over which the protruding structure 40 extends may vary based on the patient, the procedure, etc. In certain aspects, the pins 12, 14 may be threaded. The threads 40 (e.g., the protruding structure) may assist with inserting the pins 12, 14 in the bone and retaining the pins 12, 14 as implants. In certain aspects, the pins 12, 14 may define a constant threaded pitch over a substantial portion or the entire length of the pins 12, 14 or, alternatively, along segments of the pins 12, 14. In non-limiting examples, the pins 12, 14 may be threaded intramedullary pins 12, 14. It is also contemplated that the pins 12, 14 may define a variable threaded pitch without departing from the teaching herein. Further, the pins 12, 14 may have other protruding structures or components 40, such as projections, barbs, thread segments, etc., which may be used in combination with or in lieu of threads 40 to assist the pins 12, 14 in finding purchase in the bone.

The pins 12, 14 may be elongated to extend along a greater distance of the scapula. In various aspects, the pins 12, 14 may have a length between about 60 mm and about 65 mm, which may be longer than a standard implant pin. The increased length may allow the pins 12, 14 to be inserted deeper into the bone to provide increased support to the acromion A. Further, the increased length may also allow the pins 12, 14 to extend farther into the scapula S and to/into the scapular spine SS to increase the area of the bone that is reinforced. This may be advantageous for addressing and supporting multiple fracture zones as described herein. With the increased length, the pins 12, 14 may also include trimmable features 50 to remove excess length, which may have several configurations.

For example, such as the configuration illustrated in FIG. 2, the trimmable features 50 may be one or more snap features 54. The snap features 54 may have lesser widths, different construction, etc. for bending and snapping the pins 12, 14. The pins 12, 14 may be configured to snap or break at the snap features 54, leaving a portion of the pins 12, 14 implanted in the bone and removing the excess length of the pin 12, 14. The pin 12, 14 may be snapped using the bone as an anchor and/or using the aiming guide 30 (FIG. 1) to provide increased force on the removable portion. In additional or alternative examples, the pins 12, 14 may have one or more indicators that provide predefined locations for cutting or trimming the pins 12, 14. These indicators may be visual and/or tactile. In certain aspects, the indicators may be a notch, indent, lesser width, gap in the threads 40, etc. to assist with cutting the pins 12, 14 more efficiently.

The trimming device 34 (FIG. 1) may also be utilized to cut or remove excess pin 12, 14 length. The trimming device 34 may cut the pins 12, 14 at any location and may be used in combination with the trimmable features 50 or separately from the trimmable features 50. Trimming or cutting the pins 12, 14 may allow the remaining portions of the pins 12, 14 to be fully implanted in the bone.

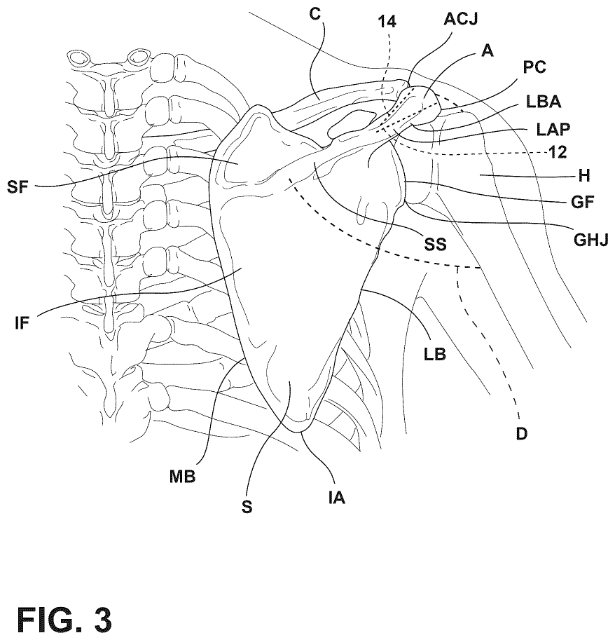

Referring to FIG. 3, the pins 12, 14 may be inserted into the intact scapula S to prophylactically address a risk of acromial fracture incidence. The pins 12, 14 may be inserted into a posterior surface of the scapula S and, more particularly, into the acromion A and toward/into the scapular spine SS. The scapular spine SS runs generally transversely across the scapula S toward a top of the scapula S to divide an infraspinous fossa IF and a supraspinous fossa SF. The infraspinous fossa IF and the supraspinous fossa SF provide locations for muscles to attach to the scapula S. The scapula S includes a lateral border LB and a medial border MB with an inferior angle IA therebetween with the lateral border LB and the medial border MB serving as muscle attachment locations. Accordingly, various muscles attach to different locations on the scapula S, which can increase stresses or forces on the bone.

The acromion A may be a bony projection extending from a lateral side of the scapular spine SS. A lateral border LBA of the acromion A may become continuous with the scapular spine SS at a lateral acromial angle LAA. The lateral border LBA of the acromion A may be above the lateral border LB of the scapula S and provides an origin site for the acromial part of the deltoid muscle. The acromion A may arch over a glenohumeral joint GHJ and articulates with a clavicle C at an acromioclavicular joint ACJ. The glenohumeral joint GHJ may be the joint between a glenoid fossa GF of the scapula S and a head of the humerus H. The glenoid fossa GF may form a shallow cavity located superiorly on the lateral border LB of the scapula S that receives the head of the humorous H. With the acromion A extending over the glenohumeral joint GHJ, injuries or procedures to the glenohumeral joint GHJ may increase stress on the acromion A, which can result in the formation of stress fractures to the acromion A.

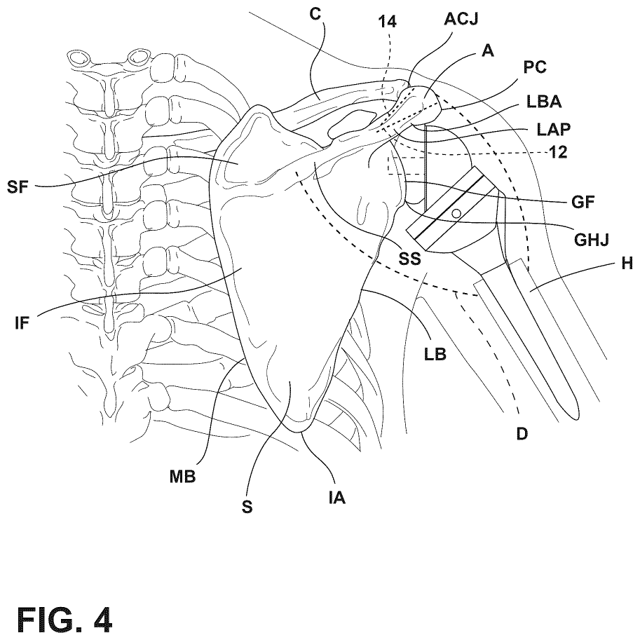

Referring now to FIGS. 3 and 4, a reverse shoulder arthroplasty (RSA) is an exemplary procedure that can result in increased stress on the acromion A. In general, shoulder replacement procedures are surgical procedures where damaged components of the shoulder area may be removed and replaced with artificial components. In a healthy shoulder, a rounded humeral head of the humerus H can move within the shallow socket of the glenoid fossa GF, as schematically illustrated in FIG. 3. In a standard shoulder replacement procedure, a ball component can be connected to the head of the humerus H and a socket can be attached to the glenoid fossa GF to replace the structure of the anatomy. In the RSA procedure, the artificial ball component may be placed on the socket side of the joint and may be attached to the scapula S, and the artificial socket component may be placed on the arm side connected to the humerus H, as illustrated schematically in FIG. 4. The RSA may be advantageous for certain injuries or conditions such as rotator cuff injuries or where attachment to the humorous H may be more difficult.

The RSA procedure can increase stress on the acromion A and can result in an increased risk of acromial fractures compared to standard shoulder replacement procedures. This increased stress can result in stress fractures of the acromion A or other scapular fractures. Acromial fractures may be more frequently encountered by patients in RSA procedures and can result in a deterioration of shoulder function. Additionally, the deltoid muscle D typically attaches to the acromion A and can increase the pressure/stress on the acromion. The deltoid D may apply a lateral/outward pulling force on the acromion, which can contribute to acromial fractures when the acromion A is weakened or in a weakened state. The acromial stress fracture may be considered a post-operative complication that can be corrected after the fracture occurs, typically by invasive plating systems to connect the bone segments.

The kit 10 described herein may provide for a prophylactic fixation of a weakened acromion A to reduce the risk of acromial stress fractures occurring after certain procedures, such as the RSA procedures. For example, during the RSA procedure, the surgical team may recognize that the scapula S is at an increased risk of developing an acromial fracture. This may be due to the anatomy of the patient, the severity of the condition, other conditions of the patient, etc. Moreover, patient demographics and comorbidities, implant configuration variables, and implant positioning variables can affect the incidence of acromion fractures. Rather than waiting until a stress fracture occurs, the pins 12, 14 may be implanted into the scapula S to provide increased support to the scapula S and reduce the likelihood of the stress fractures from occurring.

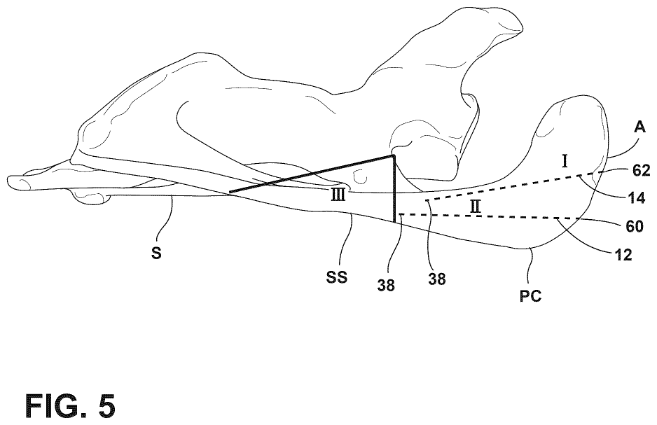

Referring to FIGS. 4 and 5, the pins 12, 14 may be inserted at the lateral border LBA of the acromion A, proximate to or at the location where the deltoid D connects to the acromion A. The pins 12, 14 may extend medially into the bone and toward/into the scapular spine SS. In certain aspects, the pins 12, 14 may be inserted parallel to the scapular spine SS anterior and posterior border, from lateral to medial on the acromion A. Based on the patient anatomy, the pins 12, 14 may be angled toward one another to reinforce an increased area of the scapula S and follow the natural anatomy of the patient. Following the natural anatomy may reduce stress caused by the pins 12, 14. The pins 12, 14 may extend at least partially across the bone and may extend at least partially across one or more weakened areas of the bone. The pins 12, 14 may maintain a spread at insertion locations 60, 62, as well as while in the bone, such as when entering the scapular spine. Alternatively, the insertion ends 38 may engage one another where the anatomy becomes narrower in the scapular spine SS.

The pins 12, 14 may be utilized to address different fracture zones. As illustrated in FIG. 5, the Levi classification can generally be utilized to identify scapular spine SS fracture patterns. A Type I fracture may represent a fracture involving the anterior position of the acromion A. A Type II fracture may represent a fracture through the anterior acromion A, posterior from the acromioclavicular joint ACJ. A Type III fracture may represent a fracture of the posterior acromion A or scapular spine SS. The most common type of fracture as a result of RSA procedures may typically be a Type II fracture. The prophylactic method described herein with the pins 12, 14 may primarily address Type I and Type Il fractures according to the Levy classification of fractures. The anatomy becomes narrower where Type III fractures typically occur, which can be more difficult to address. However, the pins 12, 14 can also be utilized for addressing all three types of fractures without departing from the teachings herein.

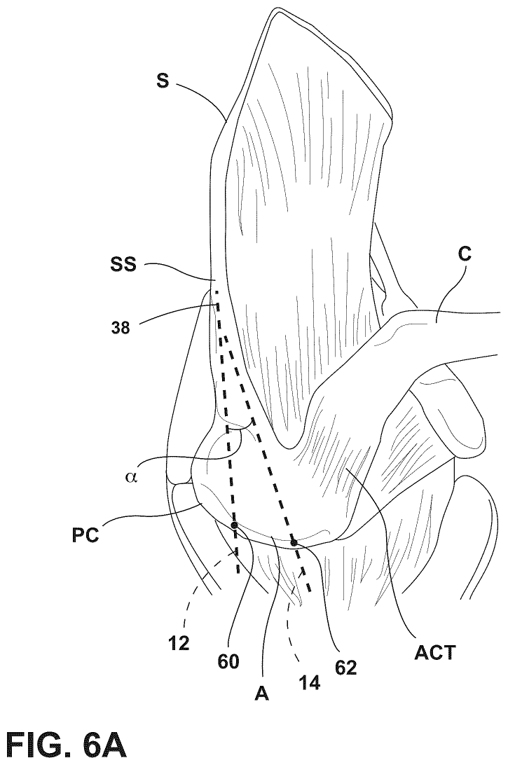

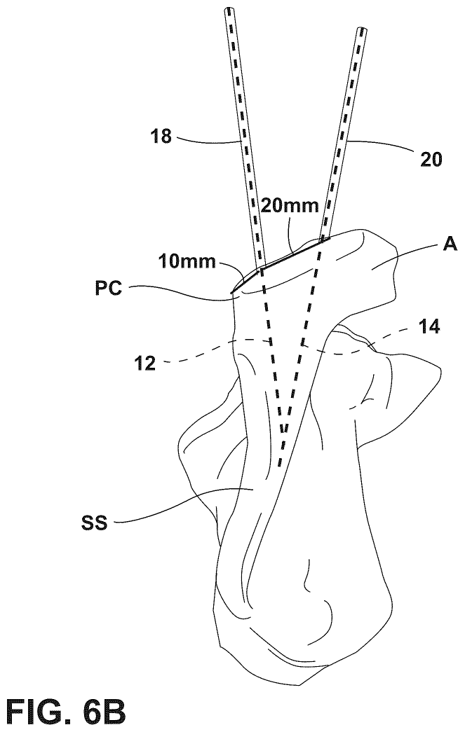

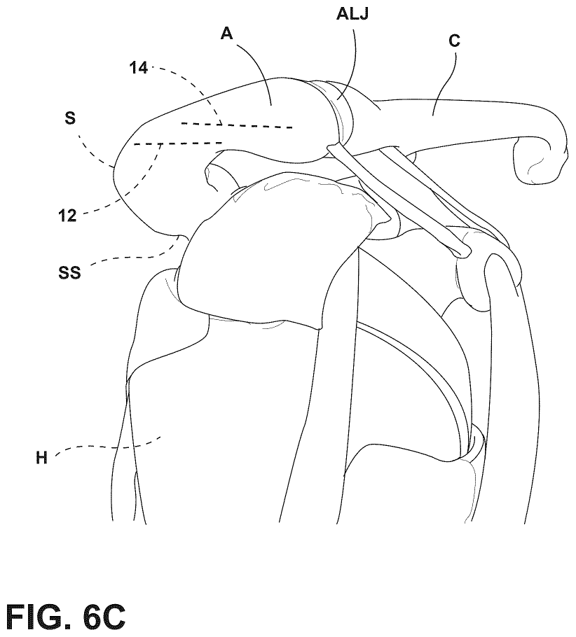

Referring now to FIGS. 6A-6C, the pins 12, 14 may be inserted into the acromion A extending medially toward and/or into the scapular spine SS to reinforce the scapula S. The pins 12, 14 may be parallel to the scapular spine SS anterior and posterior border. Reinforcing the scapula S may increase the ultimate load of the bone to reduce the likelihood of the development of an acromial fracture. Accordingly, upon insertion, the pins 12, 14 may extend transversely along the top of the scapula S. The first pin 12 may be a posterior pin 12 configured to be inserted a first predefined distance from the posterolateral corner PC of the acromion A. Generally, the first entry location 60 for the first pin 12 may be between about 5 mm and about 20 mm anterior of the most posterolateral corner PC of the acromion A. In non-limiting aspects, the first entry location 60 may be about 10 mm anterior of the most posterolateral corner PC of the acromion A. The first pin 12 may be inserted into the intact acromion A at the first entry location 60 and guided into the bone toward the scapular spine SS. The first pin 12 may be inserted to a depth of between about 50 mm and about 55 mm relative to the lateral border LBA.

The second pin 14 may be an anterior pin 14 configured to be inserted into the acromion A anterior from the first pin 12. The second entry location 62 may be located anterior to the second pin 14 and a second predefined distance from the first entry location 60. Generally, the anterior entry location 62 may be between about 10 mm and about 40 mm anterior from the first entry location 60. In a non-limiting example, the anterior entry location 62 may be about spaced from the posterior entry location 60. For example, the anterior entry location 62 may be about 20 mm anterior from the posterior entry location 60. The distance between the pins 12, 14 may vary depending on the size of the anatomy of the patient. The second pin 14 may be inserted into the acromion A at the second entry location 62 and guided into the intact bone toward the scapular spine SS. The second pin 14 may be inserted to a depth of between about 55 mm and 60 mm relative to the lateral border LBA. In a non-limiting example, the first pin 12 may be inserted to a depth of 50 mm and the second pin 14 may be inserted to a depth of 60 mm. The second pin 14 may be inserted deeper than the first pin 12 based on the anterior entry location 62 and the posterior insertion angle of the second pin 14.

The second pin 14 may be inserted coplanar to the first pin 12. Accordingly, the pins 12, 14 may extend along the same plane in the scapula S to reinforce the intact bone. The coplanar arrangement may reduce increased stress that can be caused by the inserted pins 12, 14. However, depending on the patient anatomy, the pins 12, 14 may be inserted along different planes without departing from the teachings herein.

The second pin 14 may be inserted at a posterior angle toward the first pin 12. In this regard, the entry locations 60, 62 may be a first predefined distance from one another and the insertion ends 38 of the pins 12, 14 may be a second predefined distance from one another, which may be less than the first predefined distance. This angled orientation or relationship may reinforce a greater area of the scapula S while following the anatomy that narrows medially at the scapular spine SS to reduce stress from the pins 12, 14 and fully seat the pins 12, 14 in the bone. The second pin 14 may be inserted to a greater depth due to the angled arrangement for the insertion ends 38 of the pins 12, 14 to be disposed adjacent to one another. The insertion ends 38 of the pins 12, 14 may be seated in the bone and may not extend through or out of the bone. In this regard, the cortical bone may not be breached in any direction. Accordingly, upon completion, the pins 12, 14 may extend transversely along/through the intact scapula S and be fully seated within the bone.

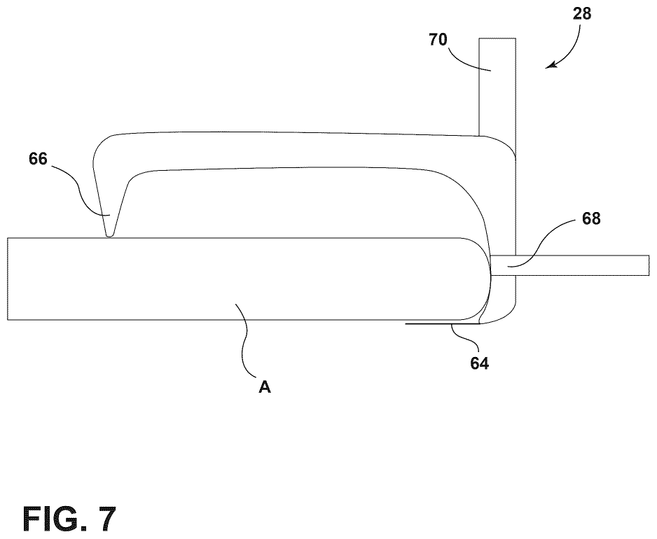

Referring to FIG. 7, the pins 12, 14 may be inserted with the use of the drill guide 28. The drill guide 28 may be able to “hook” onto the acromion A, which may assist in maintaining the position of the drill guide 28 for guiding the pins 12, 14 along the selected or predefined paths. The drill guide 28 may include a foot 64 for engaging a first surface, such as a bottom of the acromion. The size of the foot 64 may be based on the anatomy of the patient. In certain aspects, the foot 64 may be less than about 4 cm, such as about 2 cm. The drill guide 28 may include a body coupled to the foot 64 that may extend around an edge of the acromion A and over a second opposing surface, such as the top, of the acromion. The drill guide 28 may extend less than about 100 mm, such as about 50 mm along the top of the acromion A. The drill guide 28 may also include a spacer 66 coupled to the body and configured to engage the top of the acromion A, spacing the remainder of the drill guide 28 from the surface of the acromion A.

The drill guide 28 may define guide holes 68 through which a drill can be inserted to form pilot holes in the bone and/or through which the pins 12, 14 can be inserted. The foot 64 engaging the bottom of the acromion A can position the guide holes 68 proximate to the edge of the acromion A and at a predefined distance from the bottom of the acromion, which may align with the entry locations 60, 62. For example, the foot 64 can position the guide holes 68 may be less than about 5 mm, such as about 3 mm from the bottom of the acromion A. The drill guide 28 may also include an aiming point 70, which can be adjusted from superior to inferior based on, for example, soft tissue thickness. The aiming point 70 may be coupled to the body and arranged proximate to the top surface and/or the edge of the acromion A. The guide holes 68 and the aiming point 70 may assist with inserting the pins 12, 14 as well as guiding the pins 12, 14 as the pins 12, 14 are driven into the bone. The drill guide 28 may assist with maintaining proper positioning, angles, alignment, etc. of the pins 12, 14 as the pins 12, 14 are moved into the bone.

The pins 12, 14 may reinforce the intact scapula S and may reduce the likelihood that the acromial or other scapular fractures develop. In the event an acromial fraction does occur as a result of the RSA or other procedure, the implanted pins 12, 14 may assist with the setting and healing the fracture. The pins 12, 14 may assist with maintaining the natural position/shape of the scapula S from prior to the stress fracture. The pins 12, 14 may promote the bone segments in “springing” back to pre-fracture positions. This may assist with increasing union rates between the bone portions. Additionally, the pins 12, 14, may assist with maintaining alignment of the scapula S bone portions and reducing or preventing fragmenting. The implanted pins 12, 14 may also assist with isolating the fracture and reducing an increased or greater number of fractures. In this regard, the pins 12, 14 may reduce fracture incidence and may also assist with fixating bone portions together in the event of fracture occurrence after implantation. This may result in less use of large, invasive plating being utilized for acromial fractures after implantation of the pins 12, 14, which can reduce surgical repercussions to the patient.

Referring again to FIGS. 6A-7, the pins 12, 14 may be driven into the bone while connected with the shafts 22, 24. The pins 12, 14 may be inserted with the drill guide 28 and/or into pre-drilled holes formed using the drill guide 28. The pins 12, 14 may be adjusted using the driver 32 (FIG. 1) or other similar devices. Initially, with the pins 12, 14 inserted into the bone, the shafts 22, 24 and/or a portion of the pins 12, 14 may protrude from the bone. The shafts 22, 24 may be removed from the pins 12, 14, leaving the pins 12, 14 implanted in the bone. The shafts 22, 24 may be bent or otherwise moved relative to the implanted pins 12, 14 to snap or disengage the shafts 22, 24 from the pins 12, 14. In circumstances where the pins 12, 14 protrude from the bone, the pins 12, 14 may be trimmed or cut to align with the edge of the bone. In this way, the end of the implanted pins 12, 14 may form a more continuous surface with the surrounding bone to be fully seated within the bone while reducing hardware prominence. The pins 12, 14 may be trimmed at the indicators, snapped at the snap features 54, or otherwise cut to remove excess length.

The insertion of the pins 12, 14 may be prophylactic and minimally invasive. The pins 12, 14 may be inserted using a mini-open approach with a stab incision or the insertion may be percutaneous. Accordingly, larger incisions may not be utilized for this procedure, reducing post-procedure stress on the anatomy. Further, high-risk patients, such as the elderly, osteoporotic female patients, and those with inflammatory arthritis, may benefit from the prophylactic fixation of the weakened acromion. The minimally invasive technique may allow for more widespread adoption for the high- or higher-risk patients undergoing certain procedures, such as RSA procedures.

The pins 12, 14 may be inserted at locations 60, 62 proximate to where the deltoid muscle D connects to the acromion. This may assist with counteracting the pulling force applied by the deltoid D to reduce the likelihood of the acromial fracture from occurring. The pins 12, 14 may increase the ultimate load of the area of the scapula S in which the pins 12, 14 are inserted, including the attachment location for the deltoid D. The pins 12, 14 may reinforce the area prone to fracture from certain procedures (such as the acromion A) and may act as a “reinforcing bar” extending through the bone.

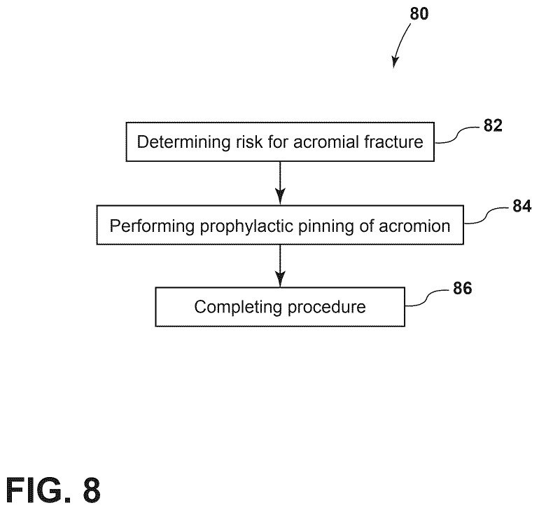

Referring to FIG. 8, as well as FIGS. 1-7, in general, the pins 12, 14 may be used in a method (80) for prophylactically reducing the likelihood of developing acromial stress fractures and/or for prophylactically reinforcing the acromion A. The surgical team may recognize that the patient is at an increased risk for developing an acromial stress fracture in response to a procedure or injury (82). The medical team may proceed with prophylactically pinning the acromion A to reinforce the acromion A and reduce the likelihood that the acromial stress fracture develops (84). Upon completion of the pinning, the procedure may be completed (86), which may include suturing the stab incisions.

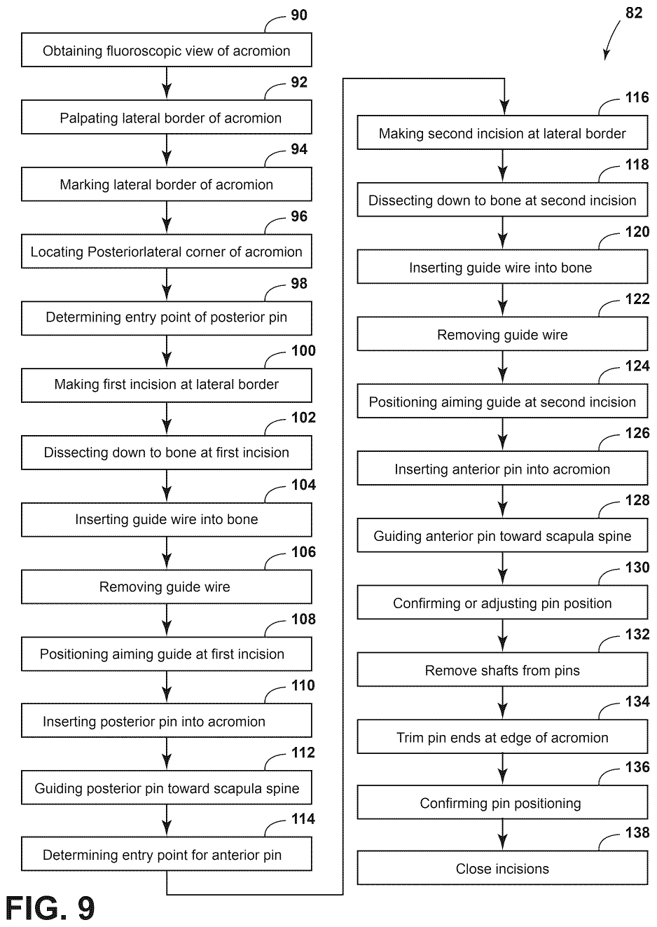

Referring to FIG. 9, as well as FIGS. 1-8, to perform the pinning (82), fluoroscopic imaging of the intact acromion A may be obtained (90). The fluoroscopic imaging may assist the medical team in locating anatomical guide points, monitoring movement of the pins 12, 14, and/or assisting with developing the procedure. For example, the aiming guide 30 and/or the drill guide 28 may be designed to guide the pins 12, 14 based on the anatomy of the patient. The fluoroscopic imaging may assist in locating the lateral border LBA, as well as determining the entry locations 60, 62, insertion depths for the pins 12, 14, and insertion angles for the pins 12, 14, providing a personalized or customized approach for the patient. The surgical team may palpate (92) and mark (94) the lateral border LBA of the acromion A.

The most posterolateral corner PC of the intact acromion A may be located or identified (96). Using the posterolateral corner PC, the first entry location 60 for the first pin 12 can be located/identified (98). The first entry location 60 may be a predefined distance anterior from the posterolateral corner PC along the acromion A. The predefined distance may depend on the anatomy of the patient and may generally be between about 5 mm and about 20 mm from the posterolateral corner PC.

When using a mini-open approach, a stab incision or percutaneous incision (100) may be made at the posterior entry location 60 at the lateral border LBA, which can be dissected down to the bone (102). This may result in a small incision that provides an opening to the acromion A. When using the drill guide 28, a percutaneous incision may be made, which may be about 1 cm in length. The percutaneous incision may be made off the posterolateral acromion A. The drill guide 28 may be inserted into the incision. A second percutaneous incision may be made over the acromion A for triangulation. When utilizing a guide wire or drill, the guide wire or drill may be inserted into the bone (104), often using the aiming guide 30 or drill guide 28. This may form a pilot hole for guiding the first pin 12 through the bone at the predefined angle. The guide wire can then be removed (106).

The aiming guide 30 and/or the drill guide 28 can be positioned on the acromion A at the posterior entry location 60 (108). In certain aspects, an additional percutaneous incision may be made over the acromion A or the same incision may be utilized for the triangulation with the drill guide 28 at the posterior entry location 60. The aiming guide 30 and/or the drill guide 28 may abut the bone and may be aimed or angled at the scapular spine SS. The angle of the aiming guide 30 and/or the direction may be determined using the fluoroscopic imaging of the patient.

The first pin assembly 18 may be inserted through the aiming guide 30 or the drill guide 28 and inserted into the bone (110). The first pin 12 may be guided toward the scapular spine SS (112). The first pin 12 may be moved using fluoroscopic guidance. In this way, the surgical team may view the insertion path of the first pin 12 to properly position the pin 12, including the angle of the first pin 12 and the depth of the first pin 12. The first pin 12 may be inserted to a depth between about 50 mm to 55 mm, aiming toward the scapular spine SS. The pin 12 may be fully contained or positioned within the intact bone with the shaft 22 extending out of the bone.

The anterior entry location 62 may be determined and located (114). The anterior entry location 62 may be between about 10 mm and about 40 mm from the posterior entry location 60 and the specific spacing may be determined based on the anatomy of the patient. A stab incision or percutaneous incision may be made at the lateral border LBA of the acromion A at the second entry location 62 (116) and dissected down to the bone (118). This may result in a second small incision that provides a second opening to the acromion A. The drill guide 28 may be adjusted and/or moved to the second entry location 62. When utilizing the guide wire or drill, the guide wire may be inserted into the bone or the drill may be used (120) to form a pilot hole for guiding the second pin 14 through the bone at the predefined angle. The guide wire may be removed (122), and the aiming guide 30 or drill guide 28 may be positioned on the acromion A at the anterior entry location 62 (124). The drill guide 28 may be positioned in the percutaneous incision and an additional percutaneous incision may be made for triangulation when using the drill guide 28.

The aiming guide 30 and/or the drill guide 28 may abut the bone and may be aimed or angled at the scapular spine SS. The angle of the aiming guide 30 and/or the drill guide 28 may be determined using the fluoroscopic imaging of the patient. The aiming guide 30 and/or the drill guide 28 may be retained in the same position to guide the second pin 14. Using fluoroscopic guidance, the second pin assembly 20 may be inserted into the acromion A through the aiming guide 30 (126) or drill guide, and the second pin 14 may be guided to a depth between about 55 mm and about 60 mm into the scapula S (128). The second pin 14 may generally be inserted coplanar to the first pin 12 and in line with the scapular spine SS. The second pin 14 may be inserted at a posterior angle with the second pin 14 being inserted toward the first pin 12. An angle, such as an acute angle, may be defined between the pins 12, 14 with the insertion ends 38 closer together than the opposing ends of the pins 12, 14 at the entry locations 60, 62. The acute angle may be between about 15° and about 32°. This angled orientation may increase an area of the scapula S that is reinforced by the pins 12, 14.

The position of the pins 12, 14 may be viewed (130), such as via fluoroscopic imaging. The aiming guide(s) 30 may be removed from the pin assemblies 18, 20 to view the position of the pins 12, 14. The shafts 22, 24 may be removed from the pins 12, 14 (132), leaving the pins 12, 14 implanted in the bone. Protruding or proud ends of the pins 12, 14 may be trimmed (134). The pins 12, 14 may be trimmed at the edge of the acromion A, typically using the pin cutter trimming device 34. The pins 12, 14 may be trimmed to be flush with the surrounding surface of the bone. Alternatively, the pins 12, 14 may be driven further into the bone using the pin driver 32 or a similar device. The positioning of the pins 12, 14 may be confirmed via fluoroscopic guidance (136). The stab incisions may be closed (138), such as via sutures, adhesive, etc., and the implanted pins 12, 14 may remain in the scapula S of the patient to reinforce the scapula S.

When using a percutaneous approach, the steps (100, 114) of forming the stab incisions and dissecting to the bone (112, 116) may be altered or omitted. Instead, with the entry locations 60, 62 identified (98, 112), smaller/less deep incisions or no incisions may be used. The aiming guide 30 may be positioned at the predefined angles (108, 122) against the skin of the patient. The pins 12, 14 may be inserted through the skin and into the bone. For both the mini-open approach and the percutaneous approach, the specific position of the entry locations 60, 62 and the insertion angles for the pins 12, 14 may be based on the anatomy of the patient, which may be determined using the fluoroscopic imaging and computed tomography (CT) scans to provide patient-specific prophylactic measures.

The pins 12, 14 may provide a prophylactic measure for reducing the likelihood of acromial stress fractures from developing in response to surgical procedures and/or injuries. The pins 12, 14 may be implanted into the bone at predefined locations and angled to extend transversely through the scapula S to reinforce the bone in which the pins 12, 14 are implanted. The pins 12, 14 may define an acute angle therebetween when implanted to extend across a greater area of the scapula S to reinforce a greater area of the scapula S.

The pins 12, 14 may increase the ultimate load for the bone, reinforcing the bone by increasing the maximum load the scapula S can withstand prior to breaking. Testing was conducted to compare weakened acromions with weakened acromions having pins (referred to as pinned acromions or pinned-weakened acromions). During testing, nine matched pairs of female fresh-frozen cadaveric right and left scapulae with a mean age 86±2 years were divided into a control group and a prophylactic fixation group for testing. The control group included both intact and weakened acromions, and the prophylactic fixation group included intact, weakened, and pinned acromions. The weakened condition consisted of an area that corresponds to a Levy Type II acromial fracture, which was centered about 1 cm in length at the level of the plane of the glenoid. To create the weakened area on the acromion, 1.8 mm cortical drill holes were drilled 2.5 mm apart, parallel to the glenoid line, within the marked weakened area. This was repeated for a total of five rows while staggering the drill holes at each adjacent line.

For the pinned acromions, two reinforcing 2.4 mm diameter Snap-Off Compression FT Pins, manufactured by Arthrex, Inc. of Naples, FL, USA, were inserted into the bone. The pins were inserted parallel to the scapular spine anterior and posterior border, from lateral to medial on the acromion. Using marked locations on the acromion, the posterior pin was inserted to a depth of 50 mm, and the anterior pin was inserted to a depth of 60 mm from the lateral acromion border. The average angle between the anterior and posterior pins inserted into the weakened acromion was 23.1±7.8 degrees.

Cyclic loading and load to failure testing were measured. For cyclic loading, SutureTape, which is manufactured by Arthrex, Inc. of Naples, FL, USA, lines were preloaded with 10 N, then cycled 10 times between 10 N to 25 N. The stiffness, hysteresis, deformation at the peak of cyclic loading, and the deformation at minimum cyclic load of the first, fifth, and last cycles were quantified and averaged. The load to failure occurred after the cyclic loading of the final condition at 60 mm/min.

Testing found that there were no significant differences in stiffness, hysteresis, displacement at peak load or displacement at minimum load when comparing the intact and weakened acromions from the control group to the pinning group. Additionally, the average stiffness of cycles 1, 5, 10 for the control-weakened and pinned-weakened acromion conditions were significantly lower than that of the respective intact acromion prior to load to failure. This corresponds to an average of an 8.7% decrease in stiffness of the weakened acromion for the control group (P=0.013) and a 6.9% decrease in stiffness of the weakened acromion for the pinning group (P=0.001).

Notably, as illustrated in Table 1 below, there was no significant difference in stiffness during load to failure testing of the weakened acromion in the control group compared to the pinned weakened (p=0.519) acromion.

TABLE 1
Load to Failure values for the weakened acromion vs. pinned-
weakened acromion with data are presented as mean ±
standard deviation and significant P-values shown in bold.

| | Weakened | Pinned-Weakened | |
|---|---|---|---|
| | Acromion | Acromion | P-Values |
| # of holes in weakened area | 46 ± 7 | 47 ± 6 | 0.560 |
| % bone removed | 47.2 ± 6.1 | 48.5 ± 6.3 | 0.518 |
| Stiffness (N/mm) | 10.3 ± 2.9 | 10.0 ± 3.1 | 0.519 |
| Yield Displacement (mm) | 8.4 ± 4.4 | 11.8 ± 4.1 | 0.004 |
| Yield Load (N) | 92.9 ± 36.4 | 122.9 ± 27.9 | 0.004 |
| Energy at Yield (N-mm) | 538.5 ± 429.0 | 877.3 ± 393.9 | <0.001 |
| Ultimate Displacement (mm) | 9.0 ± 4.6 | 12.1 ± 3.9 | 0.003 |
| Ultimate Load (N) | 95.0 ± 36.6 | 124.3 ± 28.5 | 0.005 |
| Energy at Ultimate Load (N-mm) | 592.3 ± 437.7 | 923.3 ± 386.7 | <0.001 |

In comparison to stiffness, all other failure parameters were statistically significant between the two groups. Indeed, the average displacement at yield and ultimate load, energy absorbed to yield and ultimate load, and yield and ultimate loads were significantly higher for the pinned-weakened acromion compared to the weakened acromion in the control group (p<0.001). While both the weakened acromion and the pinned-weakened acromion failed with the weakened area of bone fracturing, six of nine weakened acromions failed, whereas three of nine pinned-weakened acromions failed. The pinning of the acromion resulted in improved biomechanics, including yield displacement, yield load, energy at yield, ultimate displacement, and ultimate load, that reduced failure in the region of interest compared to the weakened acromion without pins.

In testing, the stiffness of the bone did not increase in response to insertion of the pins 12, 14, instead, the ultimate load was increased by using the pins 12, 14. Based on testing, pinning resulted in about a 30% improvement in load to failure. The pins 12, 14 may increase the maximum force the weakened bone can withstand before fracturing, as evidenced by the decrease in pinned-weakened acromions that failed during testing. The pins 12, 14 may provide a prophylactic measure to reinforce the intact scapula S that may allow the scapula S to withstand a greater ultimate load to, consequently, reduce fracture occurrence.

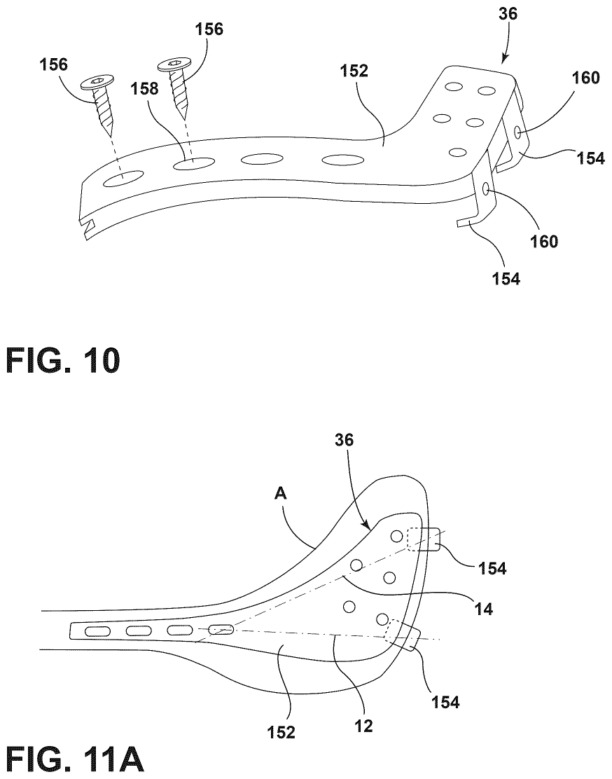

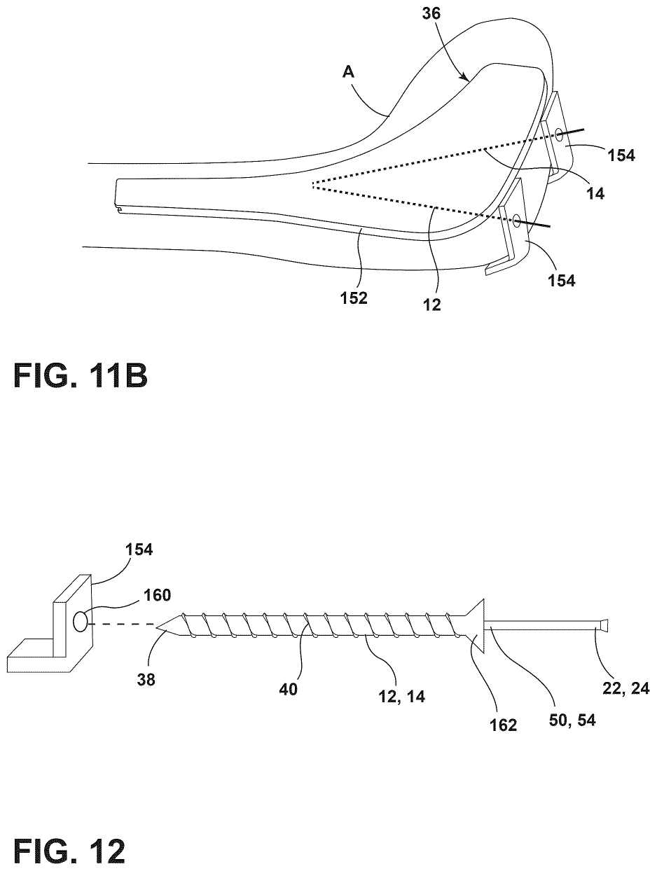

Referring now to FIGS. 10-11B, the prophylactic fixation of the acromion A may be accomplished with the pins 12, 14 alone. Additionally or alternatively, the pins 12, 14 may be utilized with a plate 36 for reinforcing the intact scapula S. The plate 36 may provide additional support for the acromion A. The pins 12, 14 may extend through the plate 36 to fix the plate 36 to the intact bone. Use of the plate 36 may depend on the patient anatomy, the weakened state of the bone, and other patient-specific factors.

The plate 36 may include a body 152 that can be manufactured to contour/match with the acromion A. In this way, the plate 36 may be personalized or customized for the patient. The plate 36 may be designed or manufactured using fluoroscopic imaging. The body 152 of the plate 36 may match the patient anatomy on axial and coronal view. This may provide increased, patient-specific reinforcement while reducing hardware/implant prominence on the acromion A. Additionally, the plate 36 matching the contour of the acromion A may contribute to the flexing of the plate 36 laterally, which may further assist with reinforcing the anatomy.

Referring now to FIGS. 10-12, the plate 36 may have hooks or tabs 154 at one end, which may be configured to engage or extend/hook over the lateral border LBA of the acromion A. Accordingly, the tabs 154 may extend over the edge of the acromion A. The tabs 154 may counteract bending movement, which can result in a biomechanically optimized plate 36 that enhances the biomechanical characteristics of the intact scapula S. The tabs 154 may also assist with positioning the plate 36 on the acromion. The plate 36 may be secured to the acromion A by the pins 12, 14 and/or additional locking features or fasteners, such as screws 156, to the lateral acromion A. The locking screws 156 may extend through apertures 158 defined along the body 152 of the plate 36.

The tabs 154 may also define apertures 160 for receiving the pins 12, 14. In this way, the plate 36 may be positioned on the intact acromion A and the pins 12, 14 may be driven through the apertures 160 in the tabs 154. The tabs 154 may be aligned with the located/determined entry locations 60, 62. In this way, imaging and palpating the anatomy may assist with determining the entry locations 60, 62, which may be utilized to manufacture or form the plate 36 with the tabs 154 aligned to the pins 12, 14. The pins 12, 14 may be driven through the tabs 154 and into the bone as described herein. The tabs 154 may not significantly impact the movement, path, angle, or depth of the pins 12, 14. As illustrated in FIG. 12, the pins 12, 14 may have a bevel 162 that may catch on the plate 36, providing an insertion depth for the pins 12, 14. The bevel 162 may be disposed adjacent to the snap-off features 50, 54, such that the bevel 162 may engage the plate 36 to assist in snapping the pins 12, 14.

The use of the plate 36 may increase the support to the acromion A while minimizing the profile of the implanted components in the patient. Additionally, the plate 36 may be patient-specific to match the contour(s) of the patient anatomy. The patient-specific approach may minimize the size of the plate 36, which can provide for a less invasive implant used to reinforce the intact scapula S to reduce the likelihood the acromial fracture develops. In the event an acromial fracture does occur, similar to the pins 12, 14, the plate 36 may assist with “springing” the anatomy back to its original position to assist with the healing process.

The plate 36 may also be utilized with a suture 164. The plate 36 may define a medial slot 166 for receiving the suture 164. The slot 166 may allow for the incorporation of a suture 164 cerclage. The suture 164 may be any practicable suture 164, such as one constructed of polyethylene for increased strength. The suture 164 may be, for example, FiberTape®, which is manufactured by Arthrex, Inc. of Naples, FL, USA, or a similar broad compression suture 164 with increased resistance. The suture 164 may be wrapped around the plate 36 and a suture 164 limb may be placed between the acromion A and the tabs 154 of the plate 36. This may be performed prior to the plate 36 being secured to the bone. The suture 164 may be passed over the plate 36 and positioned in or extended through the medial slot 166 of the plate 36 to form the cerclage. The cerclage may be secured and tensioned. The cerclage may assist with securing the plate 36 to the bone without adding significant size or additional hardware implants.

Referring again to FIGS. 8 and 10-13, the method (82) may be performed substantially similarly with the use of the plate 36 as without the plate 36. For example, with the plate 36, the fluoroscopic imaging of the intact acromion A may be obtained (90). The fluoroscopic imaging may assist the medical team in locating anatomical guide points, monitoring movement of the pins 12, 14, and/or assisting with developing the procedure. The surgical team may palpate (92) and mark (94) the lateral border LBA of the acromion A. The fluoroscopic imaging may be utilized to create the plate 36. The plate 36 may be contoured to match the acromion A, matching the anatomy on the axial and coronal views. This may allow the plate 36 to flex laterally and may allow for counter bending. The most posterolateral corner PC of the intact acromion A may be located or identified (96). Using the posterolateral corner PC, the first entry location 60 for the first pin 12 can be located/identified (98). The first entry location 60 may be a predefined distance anterior from the posterolateral corner PC along the acromion A.

A small incision may be made to insert the plate 36. The plate 36 may be positioned on the lateral acromion A with the tabs 154 hooked over the lateral border LBA of the acromion A. The locking screws 156 may be inserted through the apertures 158 to secure the plate to the acromion A. Additionally or alternatively, the suture 164 may be utilized. The suture 164 may be placed for a limb to extend between the acromion A and the tabs 154, with the sutures 164 passing over the plate 36 and through the slot 166 of the plate 36 to form the cerclage.

With the plate 36 secured via the locking screws 156 and/or the suture 164 cerclage, the method (82) may proceed substantially similarly as described with respect to FIG. 8. The aiming guide 30 and/or the drill guide 28 may be positioned and/or utilized (100-108). Pilot holes may be formed. The first pin assembly 18 may be inserted into the bone (110) through the aperture 160 of the tab 154 and confirmed (112, 114), and the second pin assembly 20 may be inserted (114-124) through the aperture 160 of the tab 154 and confirmed (120-128). The pin assemblies 18, 20 may be inserted until the bevel 162 abuts the plate 36. The pins 12, 14 may be viewed (130) and the shafts 22, 24 may be removed (132) to confirm the pin 12, 14 location (136). The methods (80, 82) may be performed in any order with steps being omitted, repeated, performed simultaneously or in order without departing from the teachings herein.

Use of the present process may provide for a variety of advantages. For example, the first and second pins 12, 14 may be implanted into and retained in the intact scapula S to increase the strength and provide support for the bone. The pins 12, 14 may reduce the incident rate of stress fractures from developing. The prophylactic fixation may result in superior biomechanical characteristics compared to the weakened acromion without the pins 12, 14. Further, the process may be minimally invasive, utilizing a percutaneous or a mini-open approach, which may reduce recovery time for the patient. Additionally, the pins 12, 14 may be inserted at an angle relative to one another to provide wider support across the acromion A/scapular spine SS and assist with mitigating or reducing stress in multiple directions. Also, the pins 12, 14 may be utilized as a prophylactic approach to reduce the likelihood or development of stress fractures, rather than a post-operative, invasive measure. Moreover, the pins 12, 14 may also assist with maintaining the natural positioning of bone portions should a fracture occur. In such cases, the pins 12, 14 may maintain alignment of the bone to assist with the union of the bone and reduce recovery time. The plate 36 may provide additional stability to the acromion A, as well as support the acromion A in combination with the pins 12, 14. Additional benefits or advantages may be realized and/or achieved.

The device disclosed herein is further summarized in the following paragraphs and is further characterized by combinations of any and all various aspects described herein.

According to an aspect of the disclosure, a method of prophylactically reducing acromial fracture incidence in response to a reverse shoulder arthroplasty may include locating a lateral border of an acromion of an intact scapula and locating a posterior entry location on the acromion at a first distance from a posterolateral corner of the acromion. A first incision may be made at the posterior entry location. A first pin may be inserted into the acromion at the posterior entry location and guided toward a scapular spine of the intact scapula. An anterior entry location on the acromion may be located. The anterior entry location may be a second distance from the posterior entry location. A second incision may be made at the anterior entry location, and a second pin into the acromion at the anterior entry location and guided toward the scapular spine of the intact scapula. The first and second pins may be trimmed at an edge of the acromion, remaining in the intact scapula.

According to an aspect of the disclosure, a method of prophylactically reducing likelihood of acromial fracture development may include locating a lateral border of an acromion of an intact scapula of a patient, as well as a posterior entry location proximate to a posterolateral corner of the acromion. A first pin can be inserted into the acromion at the posterior entry location and guided toward a scapular spine of the intact scapula. An anterior entry location may be located on the acromion spaced from the posterior entry location. A second pin may be inserted into the acromion at the anterior entry location and guided toward the scapular spine and an insertion end of the first pin. The first and second pins may be trimmed at an edge of the acromion, retaining the first and second pins in the acromion.

According to an aspect of the present disclosure, a kit for prophylactically reinforcing an acromion may include a first pin assembly including a first pin selectively coupled with a first shaft and a second pin assembly including a second pin selectively coupled with a second shaft. Each of the first and second pins may each include a protruding structure for engaging the acromion. A drill guide may include a foot configured to engage a first surface of the acromion, a body coupled to the foot and configured to extend around an edge and along a second opposing surface of the acromion, and a spacer coupled to the body. The spacer may be configured to space the body from the second opposing surface.

According to any preceding aspect of the disclosure, a method may include closing first and second incisions with first and second pins retained in an intact scapula.

According to any preceding aspect of the disclosure, a method may include obtaining an image of an intact scapula and determining first and second entry locations utilizing the image.

According to any preceding aspect of the disclosure, a method may include monitoring insertion paths of first and second pins using an image.

According to any preceding aspect of the disclosure, a step of guiding a second pin toward a scapular spine may include guiding the second pin toward an insertion end of a first pin

According to any preceding aspect of the disclosure, a method may include forming an angled relationship between first and second pins with an acute angle defined therebetween and increasing an area reinforced by the first and second pins in response to the angled relationship.

According to any preceding aspect of the disclosure, a step of inserting a first pin into an acromion may include inserting the first pin to a first depth into an intact scapula from a lateral border of the acromion. A step of inserting a second pin into the acromion may include inserting the second pin to a second depth into the intact scapula from a lateral border of the acromion. The second depth may be greater than the first depth.

According to any preceding aspect of the disclosure, a first depth may be between about 50 mm and about 55 mm, and a second depth may be between about 55 mm and about 60 mm.

According to any preceding aspect of the disclosure, a method may include providing first and second pins having a length between about 60 mm and about 65 mm.

According to any preceding aspect of the disclosure, a method may include providing first and second pins having a constant thread pitch along lengths thereof.

According to any preceding aspect of the disclosure, a first predefined distance may be between about 5 mm and about 10 mm from a posterolateral corner.

According to any preceding aspect of the disclosure, a second predefined distance may be between about 10 mm and about 40 mm.

According to any preceding aspect of the disclosure, a method may include positioning an aiming guide at an anterior entry location and guiding a first pin through the aiming guide.

According to any preceding aspect of the disclosure, a method may include positioning an aiming guide at a posterior entry location and guiding a second pin through the aiming guide.

According to any preceding aspect of the disclosure, a step of making a first incision and a step of making the second incision may include making a stab incision.

According to any preceding aspect of the disclosure, a second pin may be inserted at an acute angle toward a first pin.

According to any preceding aspect of the disclosure, a step of inserting a second pin may include inserting the second pin to a greater depth compared to a first pin.

According to any preceding aspect of the disclosure, a posterior entry location may be less than about 20 mm anterior from a posterolateral corner of an acromion.

According to any preceding aspect of the disclosure, an anterior entry location may be between about 10 mm and about 40 mm anterior from a posterior entry location.

According to any preceding aspect of the disclosure, a second pin may be inserted coplanar with a first pin.

According to any preceding aspect of the disclosure, a step of trimming first and second pins may include cutting the first and second pins.

According to any preceding aspect of the disclosure, a step of trimming first and second pins may include removing portions of the first and second pins using snap features of the first and second pins.

According to any preceding aspect of the disclosure, first and second pins may be inserted percutaneously.

According to any preceding aspect of the disclosure, a method may include making a stab incision at a posterior entry location using a mini-open approach and making a stab incision at an anterior entry location using the mini-open approach.

According to any preceding aspect of the disclosure, a method may include increasing an ultimate load of an intact scapula.

According to any preceding aspect of the disclosure, a method may include fixing a plate to the intact scapula.

According to any preceding aspect of the disclosure, a method may include positioning first and second tabs of a plate adjacent to a lateral border of an acromion, and a step of fixing the plate to an intact scapula may include inserting a first pin through a first tab and inserting a second pin through a second tab.

According to any preceding aspect of the disclosure, a step of inserting a first pin into an acromion at a posterior entry location may be performed concurrently with a step of inserting the first pin through a first tab, and a step of inserting a second pin into the acromion at an anterior entry location may be performed concurrently with a step of inserting the second pin through a second tab.

According to any preceding aspect of the disclosure, a method may include wrapping a cerclage suture around a plate and tensioning the cerclage suture.

According to any preceding aspect of the present disclosure, anterior and posterior entry locations may be disposed proximate to where a deltoid muscle connects to an acromion.

According to any preceding aspect of the present disclosure, a first surface may be a bottom surface, and a second opposing surface may be a top surface.

According to any preceding aspect of the present disclosure, a foot may be less than about 4 cm.

According to any preceding aspect of the present disclosure, a body may extend less than about 100 mm along a second opposing surface.

According to any preceding aspect of the present disclosure, a drill guide may define guide holes configured to be arranged proximate to an edge of an acromion.

According to any preceding aspect of the present disclosure, guide holes may be disposed a predefined distance from a first surface of an acromion.

According to any preceding aspect of the present disclosure, a predefined distance may be less than about 5 mm.

According to any preceding aspect of the present disclosure, a drill guide may include an aiming point coupled to a body.

According to any preceding aspect of the present disclosure, an aiming guide may be configured to adjust superiorly and inferiorly relative to an acromion.

According to any preceding aspect of the present disclosure, a kit may include a plate including a body configured to extend along an acromion and first and second tabs coupled to the body and configured to extend around an edge of an acromion.

According to any preceding aspect of the present disclosure, first and second tabs may define apertures configured to receive first and second pins, respectively.

According to any preceding aspect of the present disclosure, first and second pins may each include a bevel proximate to first and second shafts, respectively. The bevels may be configured to engage first and second tabs to define an insertion depth.

According to any preceding aspect of the present disclosure, a body of a plate may define apertures configured to receive locking features.

According to any preceding aspect of the present disclosure, a kit may include a cerclage suture configured to engage a plate. The plate may define a medial slot configured to receive the cerclage suture.

According to any preceding aspect of the present disclosure, a protruding structure on each of first and second pins may be threads. First and second pin assemblies may include a bevel proximate to the first and second pins and a snap feature proximate to the bevel, respectively.

As used herein, words of approximation such as, without limitation, “approximately,” “substantially,” or “about” refer to a condition that when so modified is understood to not necessarily be absolute or perfect but would be considered close enough to those of ordinary skill in the art to warrant designating the condition as being present. Further, the lack of such modifying terms does not otherwise require strict interpretation of the corresponding value or property. Instead, the extent to which the associated interpretation varies will depend on how great a change can be instituted and still have one of ordinary skill in the art recognize the modified feature as having the required characteristics or capabilities of the unmodified feature. Such determinations may vary considerably depending on the technological field based on the corresponding equivalency associated with the described operation or property. In general, but subject to the preceding discussion, a numerical value herein that is modified by a word of approximation such as approximately,” “substantially,” or “about” may vary from the stated value by ±0.5%, ±1%, ±2%, ±3%, ±4%, ±5%, ±10%, ±12%, or ±15%.

Any element in a claim that does not explicitly state “means” for performing a specified function or “step” for performing a specified function, should not be interpreted as a “means” or “step” clause as specified in 35 U.S.C. § 112.

It will be understood that any described processes or steps within described processes may be combined with other disclosed processes or steps to form structures within the scope of the present device. The exemplary structures and processes disclosed herein are for illustrative purposes and are not to be construed as limiting.

It is also to be understood that variations and modifications can be made on the aforementioned structures and methods without departing from the concepts of the present device, and further it is to be understood that such concepts are intended to be covered by the following claims unless these claims by their language expressly state otherwise.

The above description is considered that of the illustrated embodiments only. Modifications of the device will occur to those skilled in the art and to those who make or use the device. Therefore, it is understood that the embodiments shown in the drawings and described above are merely for illustrative purposes and not intended to limit the scope of the device, which is defined by the following claims as interpreted according to the principles of patent law, including the Doctrine of Equivalents.

The claims:

1. A method of prophylactically reducing acromial fracture incidence in response to a reverse shoulder arthroplasty, comprising:
locating a lateral border of an acromion of an intact scapula;
locating a posterior entry location on the acromion at a first predefined distance from a posterolateral corner of the acromion;
making a first incision at the posterior entry location;
inserting a first pin into the acromion at the posterior entry location;
guiding the first pin toward a scapular spine of the intact scapula;
locating an anterior entry location on the acromion that is a second predefined distance from the posterior entry location;
making a second incision at the anterior entry location;
inserting a second pin into the acromion at the anterior entry location;
guiding the second pin toward the scapular spine of the intact scapula; and
trimming the first and second pins at an edge of the acromion.

2. The method of claim 1, further comprising:
closing the first and second incisions with the first and second pins retained in the intact scapula.

3. The method of claim 1, further comprising:
obtaining an image of the intact scapula; and
determining the anterior and posterior entry locations utilizing the image.

4. The method of claim 3, further comprising:
monitoring insertion paths of the first and second pins using the image.

5. The method of claim 1, wherein the step of guiding the second pin toward the scapular spine includes:
guiding the second pin toward an insertion end of the first pin
forming an angled relationship between the first and second pins with an acute angle defined therebetween; and
increasing an area reinforced by the first and second pins in response to the angled relationship.

6. The method of claim 1, wherein the step of inserting the first pin into the acromion includes inserting the first pin to a first depth into the intact scapula from the lateral border of the acromion, and wherein the step of inserting the second pin into the acromion includes inserting the second pin to a second depth into the intact scapula from the lateral border of the acromion, and further wherein the second depth is greater than the first depth.

7. The method of claim 6, wherein the first depth is between about 50 mm and about 55 mm, and wherein the second depth is between about 55 mm and about 60 mm.

8. The method of claim 1, providing the first and second pins having a length between about 60 mm and about 65 mm.

9. The method of claim 1, wherein the first predefined distance is between about 5 mm and about 10 mm from the posterolateral corner, and wherein the second predefined distance is between about 10 mm and about 40 mm.

10. A method of prophylactically reducing a likelihood of acromial fracture development, comprising:
locating a lateral border of an acromion of an intact scapula of a patient;
locating a posterior entry location proximate to a posterolateral corner of the acromion;
inserting a first pin into the acromion at the posterior entry location;
guiding the first pin toward a scapular spine of the intact scapula;
locating an anterior entry location on the acromion spaced from the posterior entry location;
inserting the second pin into the acromion at the anterior entry location;
guiding the second pin toward the scapular spine of the intact scapula and toward an insertion end of the first pin;
trimming the first and second pins at an edge of the acromion; and
retaining the first and second pins in the acromion.

11. The method of claim 10, wherein the second pin is inserted at an acute angle toward the second pin.

12. The method of claim 10, wherein the step of inserting the second pin includes:
inserting the second pin to a greater depth compared to the first pin.

13. The method of claim 10, wherein the posterior entry location is less than about 20 mm anterior from the posterolateral corner of the acromion and wherein the anterior entry location is between about 10 mm and about 40 mm anterior from the posterior entry location.

14. The method of claim 10, wherein the second pin is inserted coplanar with the first pin.

15. The method of claim 10, wherein the first and second pins are inserted percutaneously.

16. The method of claim 10, further comprising:
making a stab incision at the posterior entry location using a mini-open approach; and
making a stab incision at the anterior entry location using the mini-open approach.

17. The method of claim 10, further comprising:
increasing an ultimate load of the intact scapula.

18. The method of claim 10, further comprising:
fixing a plate to the intact scapula; and
positioning first and second tabs of the plate adjacent to the lateral border of the acromion, wherein the step of fixing the plate to the intact scapula includes:
inserting the first pin through the first tab; and
inserting the second pin through the second tab.

19. The method of claim 18, further comprising:
wrapping a cerclage suture around the plate; and
tensioning the cerclage suture.

20. A kit for prophylactically reinforcing an acromion, comprising:
a first pin assembly including a first pin selectively coupled with a first shaft;
a second pin assembly including a second pin selectively coupled with a second shaft, wherein each of the first and second pins each include a protruding structure for engaging said acromion; and
a drill guide including:
a foot configured to engage a first surface of said acromion;
a body coupled to the foot and configured to extend around an edge and along a second opposing surface of said acromion; and
a spacer coupled to the body, wherein the spacer is configured to space the body from the second opposing surface.
